(19) United States
(12) Patent Application Publication
(10) Pub. No.: US 2007/0122813 A1
(43) Pub. Date: May 31, 2007

(54) Use of cripto-1 as a biomarker for neurodegenerative disease and method of inhibiting progression thereof

(76) Inventors: David Salomon, Frederick, MD (US); Nancy Berman, Leawood, KS (US); Edward Stephens, Kansas City, MO (US)

Correspondence Address: LEYDIG, VOIT & MAYER, LTD., TWO PRUDENTIAL PLAZA, SUITE 4900, 180 NORTH STETSON AVENUE, CHICAGO, IL, 60601-6731, US

(21) Appl. No.: 10/574,645

(22) PCT Filed: Oct. 1, 2004
(86) PCT No.: PCT/US04/32649

§ 371 Date: Aug. 10, 2006

(30) Foreign Application Priority Data
Oct. 3, 2003 (US) 60/508,750

(51) Int. Cl. C12Q 1/68; C12P 19/34; A61K 39/395

(52) U.S. Cl. 435/6; 435/91.2; 424/146.1

(57) ABSTRACT

A method of detecting a neurodegenerative disease in a mammal, which method comprises assaying the copy number of a Cripto-1 gene or the expression level of a Cripto-1 gene product in the central nervous system of the mammal, wherein an amplification of the Cripto-1 gene or an overexpression of the Cripto-1 gene product is indicative of a neurodegenerative disease in the mammal; a method of inhibiting progression of a neurodegenerative disease in a mammal, which method comprises administering to the mammal an agent that inhibits Cripto-1 in an amount effective to inhibit Cripto-1 in the central nervous system of the mammal, whereupon the progression of the neurodegenerative disease is inhibited; and an isolated or purified oligonucleotide consisting essentially of the sequence of AAGCTATGGACTGCAGGAAGATGG (SEQ ID NO: 3) or AGAAGGCAGATGCCACTAGC (SEQ ID NO: 4).

### TECHNICAL FIELD OF THE INVENTION

The present invention pertains to a method of detecting a neurodegenerative disease, a method of inhibiting progression of a neurodegenerative disease, and an isolated or purified oligonucleotide for use therein.

### BACKGROUND OF THE INVENTION

Human immunodeficiency virus (HIV-1) invades the central nervous system (CNS) within weeks after infection and causes an encephalitis (HIV-E) in approximately 25% of infected patients. The histopathology associated with this disease includes perivascular cuffing with lymphocytes and monocytes, the formation of microglial nodules and of giant cells, although the latter is not universally observed in patients with HIV-E (Navia et al., Ann. Neurol. 19: 525-535 (1986); Nebuloni et al., J. Neurovirol. 6: 46-50 (2000); Petito, Ann. Neurol. Suppl. S54-S57 (1998); and Rausch et al., J. Neuropathol. Exp. Neurol. 53: 165-175 (1994)). While it is clear that HIV-1 invades the CNS early after infection, neurologic symptoms due to HIV-1 infection, including dementia, and sensory neuropathy, usually occur at late stage when circulating CD4⁺T cells have dropped below 200 cells/μl (Price et al., Science 239: 586-592 (1988); and Singh et al., Virology 296: 39-51 (2002)). The reasons why certain patients develop HIV-E, while others do not, are not yet clear, but the particular viral strain that evolves within the patient is an important contributing factor. Another component that has not yet been well-studied is the response to viral invasion of the CNS. Release of pro-inflammatory cytolines and chemokines from infected microglia/macrophages and astrocytes has been the major mechanism to explain impaired neuronal function in the absence of direct infection of neurons. These cytokines also cause alterations in blood-brain barrier function that exposes the brain parenchyma to molecules that are toxic for neurons (Achim et al., Cur. Opin. Neurobiol. 9:221-225 (1996); Corasaniti et al., Biochem. Pharmacol. 56: 153-156 (1998); Wesselingh et al. Adv. Neuroimmunol. 4: 199-206 (1994); Wesselingh et al., J. Neuroimmunol. 74: 1-8 (1997); and Wesselingh et al., Curr. Opin. Neural. 14: 375-379 (2001)). Cytokine expression has been observed in acquired immunodeficiency syndrome (AIDS), but possible expression of neuroprotective factors has not been evaluated.

Several non-human primate models have been used to gain insight into the neuropathogenesis of HIV-1. The simian immunodeficiency virus (SIV)_{mac}/macaque model has provided much useful information on the early events of neuroinvasion. Studies have shown that both T cell tropic and neuropathogenic stains of SIV_{mac} enter the CNS early after inoculation, and that development of simian immunodeficiency virus-encephalitis (SIV-E) correlates with viral loads in the cerebrospinal fluid (CSF) (Zink et al., J. Virol. 73: 10480-10488 (1999)). In addition to the SIV_{mac}/macaque model, investigators also have used the chimeric simian human immunodeficiency virus (SHIV), which contains the tat, rev, vpu, and env of HIV-1 in a genetic background of SIV_{mac}239. Pathogenic SHIVs have been derived in several laboratories and are associated with high virus burdens, rapid loss of circulating CD4⁺T cells and depletion of T cell rich areas of the thymus, lymph nodes and spleen (Joag et al., J. Virol. 70: 3189-3197 (1996); Luciw et al., Virology 263: 112-127 (1999); Raghavan et al., Neuropathol. Appl. Neurobiol. 25: 285-294 (1999); and Shibata et al., J. Infect. Dis. 176: 362-373 (1997)). Macaques inoculated with pathogenic SHIVs generally succumb to their disease within 6-8 months, and similar to SIV_{mac} model, SHIV-inoculated macaques can develop neurological disease and a neuropathology that is similar to SIV-E (Liu et al., Virology 260: 295-307 (1999); McCormick et al., Virology 272: 112-126 (2000); and Raghavan et al., Brain Pathol. 7: 851-861 (1997)).

Despite the research currently taking place in this area, there remains a need in the art for the identification of genes that are differentially expressed in mammals that are infected with HIV-1 in the CNS, as well as those that are differentially expressed in other neurodegenerative diseases. In this regard, there remains a need in the art for a method of detecting a neurodegenerative disease through assaying the expression level of specific genes. The present invention provides such a method. This and other objects and advantages of the invention, as well as additional inventive features, will be apparent from the description of the invention provided herein.

### BRIEF SUMMARY OF THE INVENTION

The present invention provides a method of detecting a neurodegenerative disease in a mammal. The method comprises assaying the copy number of a Cripto-1 gene or the expression level of a Cripto-1 gene product in the central nervous system of the mammal. In this method, an amplification of the Cripto-1 gene or an overexpression of the Cripto-1 gene product is indicative of a neurodegenerative disease in the mammal.

The present invention also provides a method of inhibiting progression of a neurodegenerative disease in a mammal. The method comprises administering to the mammal an agent that inhibits Cripto-1 in an amount effective to inhibit Cripto-1 in the central nervous system of the mammal. Through this method, the progression of the neurodegenerative disease is inhibited.

Further provided by the present invention is an isolated or purified oligonucleotide consisting-essentially of the sequence of AAGCTATGGACTGCAGGAAGATGG (SEQ ID NO: 3) or AGAAAGGCAGATGCCAACTAGC (SEQ ID NO: 4).

### DETAILED DESCRIPTION OF THE INVENTION

The present invention provides a method of detecting a neurodegenerative disease in a mammal. The method comprises assaying the copy number of a Cripto-1 gene or the expression level of a Cripto-1 gene product in the central nervous system of the mammal. In this method, an amplification of the Cripto-1 gene or an overexpression of the Cripto-1 gene product is indicative of a neurodegenerative disease in the mammal.

As used herein, the term “neurodegenerative disease” refers to any disease, disorder, abnormal condition, or malady of the central nervous system. Neurodegenerative diseases include, for instance, NeuroAIDS, Alzheimer's disease, multiple sclerosis, amyotrophic lateral sclerosis (ALS), Parkinson's disease, encephalitis, stroke, trauma (e.g., head trauma), and the like. With respect to the present invention, the neurodegenerative disease is preferably NeuroAIDS, Alzheimer's disease, multiple sclerosis, ALS, Parkinson's disease, or encephalitis. More preferably, the neurodegenerative disease is NeuroAIDS.

The Cripto-1 gene, also known in the art as the Teratocarcinoma-derived Growth Factor-1 (TDGF-1) gene, encodes a protein consisting of 188 amino acids. The Cripto-1 protein is a member of the Epidermal Growth Factor-cysteine rich motif (EGF-CFC) family of proteins. The coding sequence of the human Cripto-1 gene and the amino acid sequence of the encoded gene product, i.e., the encoded protein, are publicly available at the National Center for Biotechnology Information (NCBI) website as GenBank Accession No. M96955 (SEQ ID NO: 1) and AAA61134 (SEQ ID NO: 2), respectively.

The term “amplification” as used herein refers to an increase in the copy number of chromosomal sequences, i.e., genes. The. term “overexpression” as-used herein means an increase in the level of gene product, e.g., protein or nucleic acid mnolecule (e.g., MRNA), either of which is encoded by the Cripto-1 gene. The term “nucleic acid molecule” can be any nucleic acid molecule, e.g., RNA (e.g., MRNA) and cDNA, as long as it is encoded by the Cripto-1 gene.

Methods of determining whether or not a mammal has an amplification of a particular gene are known in the art. Suitable methods include, for instance, Polymerase Chain Reaction (PCR), microarray analysis, in situ hybridization, and Southern blotting, some of which are described in Sambrook et al., Molecular Cloning: A Laboratory Manual 2^{nd} ed., Cold Spring Harbor Press, Cold Spring Harbor, N.Y., 1989. In such methods, an oligonucleotide probe designed to hybridize selectively to the gene of which an amplification is being determined (i.e., the Cripto-1 gene) is added to a sample containing genomic DNA obtained from the mammal. The oligonucleotide probe and the genomic DNA of the sample are incubated under conditions that permit selective hybridization. Preferably, the hybridization is done under high stringency conditions. By “high stringency conditions,” it is meant that the probe specifically hybridizes to target sequences of the genomic DNA in an amount that is detectably stronger than non-specific hybridization. High stringency conditions, then, would be conditions, which would distinguish a polynucleotide with an exact complementary sequence of the target sequences of the genomic DNA from those sequences containing only a few small regions (e.g., 3-10 bases) with exact complementary sequence of the targets of the genomic DNA. Such small regions of complementarity are more easily melted than a full-length complement of 14-17 or more bases and high stringency hybridization makes them easily distinguishable. High stringency conditions would include, for example, low salt and/or high temperature conditions, such as provided by about 0.02-0.1 M NaCl or the equivalent, at temperatures of about 50-70° C. Such high stringency conditions tolerate little, if any, mismatch between the probe and the target sequences of the genomic DNA and are particularly suitable for detecting amplifications of genomic sequences. It is generally appreciated that conditions can be rendered more stringent by the addition of increasing amounts of formamide.

After incubating the oligonucleotide probe and the genomic DNA obtained from the mammal, the complex comprising the oligonucleotide probe hybridized to the genomic DNA, or portion thereof, is amplified before detection. Amplification can be achieved through template-dependent amplification of the genomic DNA sequence that is adjacent to the nucleotide sequence to which the oligonucleotide probe hybridizes. Various template-dependent processes for amplifyg such DNA sequence are Ikown in the art, a number of which are described in Sambrook et al. (1998), supra. One of the best-known processes is PCR. In this method, the complex is contacted with one or more enzymes that facilitate template-dependent nucleic acid synthesis. Preferred enzymes include, for example, DNA polymerases, such as T4 DNA polymerase and TaQMan DNA polymerase (Applied Biosystems, Foster City, Calif.). Multiple rounds of amplification, also referred to as “cycles,” are conducted until a sufficient amount of amplification product, or amplicons, is produced.

Other methods for amplification of the genomic DNA sequence include the ligase chain reaction (LCR), which is disclosed in U.S. Pat. No. 4,883,750; isothermal amplification, in vihich- restriction endonucleases and ligases are used to achieve the amplification of molecules that contain nucleotide 5′-[α-thio]-triphosphates in one strand (Walker et al., Proc. Natl Acad. Sci. USA 89: 392-396 (1992)); strand displacement amplification (SDA), which involves multiple rounds of strand displacement and synthesis, i.e., nick translation, and repair chain reaction (RCR), which involves annealing several probes throughout a region targeted for amplification, followed by a repair reaction in which only two of the four bases are present. The other two bases can be added as biotinylated derivatives for easy detection. Target-specific sequences also can be detected using a cyclic probe reaction (CPR). In CPR, a probe having 3′ and 5′ sequences of non-specific DNA and a middle sequence of specific RNA is hybridized to DNA, which is present in a sample. Upon hybridization, the reaction is treated with RNase H, and the products of the probe are identified as distinctive products, which are released after digestion. The original template is annealed to another cycling probe, and the reaction is repeated. A number of other amplification processes are contemplated; however, the invention is not limited as to which method is used.

Following amplification of the genomic DNA sequence, it can be desirable to separate the amplicons from the oligonucleotide probe for the purpose of determining whether specific amplification has occurred. In one embodiment, the amplicons are separated by agarose, agarose-acrylamide or polyacrylamide gel electrophoresis using standard methods. See Sambrook et al. (1989), supra. Alternatively, chromatographic techniques can be employed to effect separation. There are many kinds of chromatography that can be used in the context of the present inventive methods, e.g., adsorption, partition, ion-exchange and molecular sieve, and many specialized techniques for using them including column, paper, thin-layer and gas chromatography exist; (Freifelder, Physical Biochemistry Applications to Biochemistry and Molecular Biology, 2^{nd} Ed., Wm. Freeman and Co., New York, N.Y. (1982)).

Amplicons must be visualized in order to confirm that hybridization of the oligonucleotide probe with the genomic DNA occurred. One typical visualization method involves staining of a gel with ethidium bromide and visualization under UV light. Alternatively, if the amplicons are integrally labeled with radio-, colorimetrically-, or fluorometrically-labeled nucleotides, the amplicons then can be exposed to x-ray film or visualized under the appropriate stimulating spectra, following separation. The oligonucleotide probe that hybridizes can, alternatively, be radio-, calorimetrically-, or fluorometrically-labeled.

Alternatively, visualization of the amplicons can be achieved indirectly. Following separation of the amplicons from the oligonucleotide probe, another oligonucleotide probe is brought into contact with the amplicons. This other probe can be conjugated to a chromophore or can be radiolabeled. In another embodiment, the other probe is conjugated to a binding partner, such as an antibody or biotin, where the other member of the binding pair carries a detectable moiety (i.e., a label).

One example of the foregoing is described in U.S. Pat. No. 5,279,721, which discloses an apparatus and method for the automated electrophoresis and transfer of nucleic acids. The apparatus permits electrophoresis and blotting without external manipulation of the gel and is ideally suited to carrying out methods according to the present invention.

In the foregoing method of determining whether or not a mammal has an amplification of the Cripto-1 gene, it may be desirable to carry out the methods with a control, wherein the control is a sample containing genomic DNA of a mammal that is known not to have a neurodegenerative disease. In this manner, the copy number of the genes of the test mammal can be directly-compared to that of the control.

Methods of determining whether or not a mammal has an overexpression of a Cripto-1 gene product (protein or a nucleic acid molecule) are also known in the art. Suitable methods include, for instance, Western blotting, in the case that an overexpression of a protein is being determined, and Northern blotting, Reverse transcription-PCR (RT-PCR), and Real-Time PCR, in the case that an overexpression of a RNA or cDNA is being determined. Such methods are described in Sambrook et al. (1998), supra; and U.S. Pat. No. 5,654,140.

In a preferred embodiment of the present inventive method, the method comprises non-quantitative RT-PCR. By “non-quantitative” is meant that the RT-PCR does not determine the actual quantity of nucleic acid molecules expressed in the central nervous system of the mammal. An example of non-quantitative RT-PCR is described herein as Example 2. Preferably, the RT-PCR is carried out with oligonucleotide probes consisting essentially of nucleotide sequences of AAGCTATGGACTGCAGGAAGATGG (SEQ ID NO: 3) and AGAAAGGCAGATGCCAACTAGC (SEQ ID NO: 4). It will be understood that the oligonucleotide probes described above are limited inasmuch as any oligonucleotide having any nucleotide sequence can be used as long as the oligonucleotide is hybridizable to the Cripto-1 gene of the genomic DNA.

In this regard, the present invention also provides an isolated or purified oligonucleotide consisting essentially of the sequence of AAGCTATGGACTGCAGGAAGATGG (SEQ ID NO: 3) or AGAAAGGCAGATGCCAACTAGC (SEQ ID NO: 4). The term “isolated” as used herein is-defined as having been removed from its natural environment. The term “purified” as used herein is defined as having removed some or all other constituents. The term “oligonucleotide” as used herein is defined as a polymer of DNA or RNA, (i.e., a polynuleotide), which can be single-stranded or double-stranded, synthesized or obtained from natural sources, and which can contain natural, non-natural or altered nucleotides and can contain natural, non-natural or altered intemucleotide linkages. With respect to the isolated or purified oligonucleotides of the present invention, it is preferred that no insertions, deletions, inversions, and/or substitutions are present in the oligonucleotide. However, it may be suitable in some instances for the isolated oligonucleotides of the present invention to comprise one or more insertions, deletions, and/or substitutions. It is, furthermore, preferred that the isolated oligonucleotides of the present invention are synthesized, single-stranded polymers of DNA.

Alternatively or additionally, the method comprises using a cDNA array. The term “cDNA array” as used herein refers to any solid support containing a plurality of different cDNAs organized into a multi-dimensional matrix or array. The cDNA array can be any cDNA array provided that it contains an oligonucleotide that specifically hybridizes to a nucleic acid molecule encoding a Cripto-1 gene product, e.g., the Cripto-1 gene itself, a Cripto-1 MRNA, or a Cripto-1 protein. cDNA arrays can be purchased, as they are commercially available from companies, such as Clontech (Palo Alto, Calif.).

When determining whether or not a mammal has an overexpression of a protein encoded by the Cripto-1 gene, various assays (i.e., immunobinding assays) are contemplated. The various useful immunodetection assays have been described in Nakamura et al., Handbook of Experimental Immunology, 4^{th} ed., Wol. 1, Chapter 27, Blackwell Scientific Publ., Oxford, 1987 and include Western blotting, enzyme-linked immunosorbent assay (ELISA), and radioimmunoassay. Immunobinding assays specific for Cripto-1 are described in references, such as International Patent Application Nos. WO 02/088170 and WO 02/059620.

In general, the immunobinding assays involve obtaining a sample containing the protein encoded by the Cripto-1 gene, a peptide fragment thereof, or an antibody that specifically binds to the protein or peptide fragment thereof, and contacting the sample with an antibody that specifically binds to the protein, peptide or antibody under conditions effective to allow the formation of immunocomplexes. Any suitable antibody can be used in conjunction with the present invention, such that the antibody is specific for the protein or peptide fragment thereof encoded by the Cripto-1 gene or antibody thereto. Such antibodies can be made in accordance with those methods of making antibodies known in the art. See, for instance, Harlow et al., Antibodies: A Laboratory Manual, Cold Spring Harbor Publishers, Cold Spring Harbor, N.Y., 1988. Also, Cripto-1 antibodies are described in references, such as U.S. Pat. No. 5,654,140. Alternatively, fragments of the antibody can be used as long as the fragment specifically binds to the protein encoded by the Cripto-1 gene. Such fragments are known in the art to include, for instance, F(ab)₂′ fragments, single chain anitibody variable region fragment (ScFv) chains, and the like.

The immunobinding assays for use in the present invention include methods of detecting or quantitating the immune complexes formed upon incubating the sample with the antibody. In general, the detection of immune complexes is well-known in the art and can be achieved through the application of numerous approaches. These methods are generally based upon the detection of a label or marker, such as any radioactive, fluorescent, biological or enzymatic tags or labels of standard use in the art. U.S. Patents concerning the use of such labels include U.S. Pat. Nos. 3,817,837; 3,850,752; 3,939,350; 3,996,345; 4,277,437; 4,275,149 and 4,366,241. Of course, additional advantages can be realized by using a secondary binding ligand, such as a second antibody or a biotin/avidin ligand binding arrangement, as is known in the art.

The antibody used to form the immune complexes can, itself, be linked to a detectable label, wherein one would then simply detect this label, thereby allowing the presence of or the amount of the primary immune complexes to be determined.

Alternatively, the first added component that becomes bound within the primary immune complexes can be detected by means of a second binding ligand that has binding affinity for the first antibody. In these cases, the second binding ligand is, itself, often an antibody, which can be termed a “secondary” antibody. The primary immune complexes are contacted with the labeled, secondary binding ligand, or antibody, under conditions effective and for a period of time sufficient to allow the formation. of secondary immune complexes. The secondary immune complexes are then washed to remove any non-specifically bound labeled secondary antibodies or ligands, and the remaining label in the secondary immune complexes is then detected.

Further methods include the detection of primary immune complexes by a two-step approach. A second binding ligand, such as an antibody, that has binding affinity for the first antibody is used to form secondary immune complexes, as described above. After washing, the secondary immune complexes are contacted with a third binding ligand or antibody that has binding affinity for the second antibody, again under conditions effective and for a period of time sufficient to allow the formation of immune complexes (tertiary immune complexes). The third ligand or antibody is linked to a detectable label, allowing detection of the tertiary immune complexes thus formed. A number of other assays are contemplated; however, the invention is not-limited as to which method is used.

For purposes of the present inventive methods, the mammal can be any mammal, including, but not limited to, mammals of the order Rodentia, such as mice, the order Logomorpha, such as rabbits, the order Carmivora, including Felines (cats) and Canines (dogs), the order Artiodactyla, including Bovines (cows) and Swines (pigs), the order Perssodactyla, including Equines (horses), the order Primates, Ceboids, or Simoids (monkeys) or of the order Anthropoids (humans and apes). An especially preferred mammal is the human.

In the present inventive method, the copy number of a Cripto-1 gene or the expression level of a Cripto-1 gene product is assayed from a cell, tissue, fluid, organ, or part thereof of the central nervous system. The central nervous system includes, for example, the brain, spinal cord, ganglia, nerves, and cerebrospinal fluid. Preferably, the expression level of the Cripto-1 gene product is assayed from cerebrospinal fluid obtained from the mammal.

The present invention also provides a method of inhibiting progression of a neurodegenerative disease in a mammal. The method comprises administering to the mammal an agent that inhibits Cripto-1 in an amount effective to inhibit Cripto-1 in the central nervous system of the mammal. Through this method, the progression of the neurodegenerative disease is inhibited. For purposes of the present invention, the phrase “agent that inhibits Cripto-1 ” refers to any chemical compound, natural or synthetic, that inhibits the function of the protein encoded by the Cripto-1 gene. As generally known by one of ordinary skill in the art, the function of the Cripto-1 protein is to stimulate growth and regulate cellular differentiation through Nodal signalling. In this regard, an agent that inhibits Cripto-1 will inhibit Nodal signaling and SMAD-induced gene activiation. As used herein, the term “inhibit,” and words stemming therefrom, do not necessarily imply 100% or complete inhibition. Rather, there are varying degrees of inhibition of which one of ordinary skill in the art recognizes as having a potential benefit or therapeutic effect. In this regard, agents that inhibit the Cripto-1 protein can induce any level of inhibition. Desirably, the agents that inhibit Cripto-1 can inhibit at least 10% of the function or activity of the Cripto-1 protein in the absence of any agents that inhibit the Cripto-1 protein. It is more preferred that the agents that inhibit Cripto-1 achieve at least 50% inhibition. Most preferably, the agent that inhibits Cripto-1 inhibits 90% or more of the activity of the Cripto-1 protein in the absence of any agents that inhibit Cripto-1.

For purposes of the present inventive method of inhibiting the progression of a neurodegenerative disease, any agent that inhibits Cripto-1 can be employed. The agent can be, for instance, a peptide that specifically binds to a Cripto-1 protein or a growth factor inhibitor. Such agents are known in the art (see, for instance, U.S. Pat. No. 5,654,140). The agent that inhibits Cripto-1 can be a mutant Cripto-1 protein, such as one of those described in International Patent Application No. WO 02/22808.

Alternatively, the agent that inhibits Cripto-1 can be an isolated or purified oligonucleotide that can hybridize to a nucleic acid molecule encoding the protein, such that administration of the oligonucleotide will result in the inhibition of the expression of the protein. The oligonucleotide can be of any length, comprising any number of nucleotides, as long as it can hybridize to the nucleic acid molecule encoding the protein. Preferably, the oligonucleotide that can hybridize is at least 18 nucleotides in length. Furthermore, the oligonucleotide can be of any nucleotide sequence as long as it can hybridize to the nucleic acid molecule in a manner sufficient to inhibit the expression of the protein. While it is likely that many other oligonucleotides having different sequences are suitable for use in the present inventive methods, the oligonucleotide preferably comprises, consists essentially of, or consists of the nucleotide sequence of SEQ ID NO: 3 or SEQ BD NO: 4.

A variety of techniques used to synthesize the present inventive oligonucleotides are known in the art. See, for example, Sambrook et al., 1989, supra; and Lemaitre et al., Proc. Natl. Acad. Sci. USA 84: 648-652 (1987). The oligonucleotides can alternatively by synthesized commercially by companies, such as Eurogentec, Belgium.

The agent that inhibits Cripto-1 can, alternatively, be an antibody, or fragment thereof, that binds specifically to the Cripto-1 protein. Antibodies suitable for use in the present inventive method of inhibiting progression of a neurodegenerative disease can be synthesized by methods of making antibodies that are known in the art. See, for example, Harlow et al., Antibodies: A Laboratory Manual, Cold Spring Harbor Publishers, Cold Spring Harbor, N.Y., 1988. Fragments of antibodies that bind to the Cripto-1 protein are also suitable for use. The fragment can be any fragment that binds specifically to the protein. The fragment can include, for instance, an F(ab₂)′ fragment. One of ordinary skill in the art recognizes that, in general, antibodies, and fragments thereof, will bind to the Cripto-1 protein and prevent its activity by preventing a substrate or another protein from binding to the Cripto-1 protein, wherein the binding of the substrate or other protein is necessary for the function of the Cripto-1 protein.

Methods of identifying an agent that inhibits Cripto-1 are known in the art. For instance, an agent that is suspected to have Cripto-1-inhibiting activity could be administered at varying doses to cells expressing Cripto-1 and subsequently tested for Cripto-1 activity. Methods of testing Cripto-1 activity are known in the art and are described in references, such as Bianco et al., Mol. Cell. Bio. 22: 2586-2597 (2002) and Bianco et al., Cancer Res. 63: 1192-1197 (2003). The degree to which the Cripto-1 activity is inhibited by the agent suspected to have Cripto-1-inhibiting activity in a dose-dependent manner can be compared to the degree to which the Cripto-1 activity was inhibited in cells that were not administered any agent (a negative control) and/or that were administered an agent that is known to have Cripto-1-inhibiting activity (a positive control). Furthermore, the agent that was being tested for Cripto-1-inhibiting activity in the above-described in vitro assay can additionally or alternatively be tested for Cripto-1-inhibiting activity in an animal. In this instance, the agent that was tested is administered at varying doses to a set of animals, each receiving a different dose of the agent. After regularly administering the agent to the animals, specimen (e.g., cells or tissues) that contain Cripto-1 are obtained from the animals and are tested for Cripto-1 activity. Methods of testing Cripto-1 activity may be tested in the same manner as in the in vitro assay. As in the in vitro assay, the degree to which the Cripto-1 activity is inhibited by the agent suspected to have Cripto-1-inhibiting activity in a dose-dependent manner can be compared to the degree to which the Cripto-1 activity was inhibited in an animal that was not administered any agent (a negative control) and/or that was administered an agent that is known to have Cripto-1-inhibiting activity (a positive control).

Agents that inhibit Cripto-1 that are useful in the present inventive methods can be in the form of a salt, which is preferably a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example p-toluenesulphonic acid.

Agents that inhibit Cripto-1 that can be used in the present inventive methods, can be formed as a composition, such as a pharmaceutical composition. Pharmaceutical compositions containing the agent that inhibit Cripto-1 can comprise more than one active ingredient, such as more than one type of inhibitor of the protein, e.g., a composition comprising an antibody specific for Cripto-1 and an isolated or purified oligonucleotide having the nucleotide sequence of SEQ ID NO: 3 or SEQ ID NO: 4. The pharmaceutical composition can alternatively comprise an inhibitor of the protein in combination with another pharmaceutically active agent or drug.

The composition comprising the agent that inhibits Cripto-1 preferably comprises a carrier. The carrier can be any suitable carrier. Preferably, the carrier is a pharmaceutically acceptable carrier. With respect to pharmaceutical compositions, the carrier can be any of those conventionally used and is limited only by chemico-physical considerations, such as solubility and lack of reactivity with the active compound(s), and by the route of administration. It will be appreciated by one of ordinary skill in the art that, in addition to the following described pharmaceutical composition, the compounds and inhibitors of the present inventive methods can be formulated as inclusion complexes, such as cyclodextrin inclusion complexes, or liposomes.

The pharmaceutically acceptable carriers described herein, for example, vehicles, adjuvants, excipients, and diluents, are well-known to those skilled in the art and are readily available to the public. It is preferred that the pharmaceutically acceptable carrier be one which is chemically inert to the active agent(s) and one which has no detrimental side effects or toxicity under the conditions of use.

The choice of carrier will be determined in part by the particular agent, as well as by the particular method used to administer the agent that inhibits Cripto-1. Accordingly, there are a variety of suitable formulations of the pharmaceutical composition of the present inventive methods. The following formulations for oral, aerosol, parenteral, subcutaneous, intravenous, intramuscular, interperitoneal, rectal, and vaginal administration are exemplary and are in no way limiting. One skilled in the art will appreciate that these routes of administering the agent or composition comprising the agent are known, and, although more than one route can be used to administer a particular agent, a particular route can provide a more immediate and more effective response than another route.

Injectable formulations are among those formulations that are preferred in accordance with the present invention. The requirements for effective pharmaceutical carriers for injectable compositions are well-known to those of ordinary skill in the art (see, e.g., Pharmaceutics and Pharmacy Practice, J. B. Lippincott Company, Philadelphia, Pa., Banker and Chalners, eds., pages 238-250 (1982), and ASHP Handbook on Injectable Drugs, Toissel, 4th ed., pages 622-630 (1986)).

Topical formulations are well-known to those of skill in the art. Such formulations are particularly suitable in the context of the present invention for application to the skin.

Formulations suitable for oral administration can consist of (a) liquid solutions, such as an effective amount of the agent that inhibits Cripto-1 dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant. Capsule forms can be of the ordinary hard- or soft-shelled gelatin type containing, for example, surfactants, lubricants, and inert fillers, such-as-lactose, sucrose, calcium phosphate, and corn starch. Tablet forms can include one or more of lactose, sucrose, mannitol, corn starch, potato starch, algnic acid, microcrystalline cellulose, acacia, gelatin, guar gum, colloidal silicon dioxide, croscarmellose-sodium, talc,.magnesium stearate, calcium stearate, zinc stearate, stearic acid, and other excipients, colorants, diluents, buffering agents, disintegrating agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

The agent that inhibits Cripto-1, alone or in combination with another agent that inhibits Cripto-1 and/or with other suitable components, can be made into aerosol formulations to be administered via inhalation. These aerosol formulations can be placed into pressurized acceptable propellants, such as dichlorodifluoromethane, propane, nitrogen, and the like. They also can be formulated as pharmaceuticals for non-pressured preparations, such as in a nebulizer or an atomizer. Such spray formulations also can be used to spray mucosa.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The agent that inhibits Cripto-1 can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, dimethylsulfoxide, glycerol ketals, such as 2,2-dimethyl-1,3-dioxolane4-methanol, ethers, such as poly(ethyleneglycol) 400, an oil, a fatty acid, a fatty acid ester or glyceride, or an acetylated fatty acid glyceride, with or without the addition of a pharmaceutically acceptable surfactant, such as a soap or a detergent, a suspending agent, such as pectin, carbomers, methylcellulose, hydroxypropyhnethylcellulose, carboxymethylcellulose, emulsifying agents and/or other pharmaceutical adjuvants.

Oils, which can be used in parenteral formulations, include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and. polyoxyethylenepolypropylene copolymers, (d) amphoteric detergents such as, for example, alkyl-b-aminopropionates, and 2-alkyl-imidazoline quaternary ammonium salts, and (e) mixtures thereof.

The parenteral formulations will typically contain from about 0.5% to about 25% by weight of the active ingredient in solution. Preservatives and buffers can be used. In order to minimize or eliminate irritation at the site of injection, such compositions may contain one or more nonionic surfactants having a hydrophile-lipophile balance (HLB) of from about 12 to about 17. The quantity of surfactant in such formulations will typically range from about 5% to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

Additionally, the agent that inhibits Cripto-1, or compositions comprising such an agent that inhibits Cripto-1, can be made into suppositories by mixing with a variety of bases, such as emulsifying bases or water-soluble bases. Formulations suitable for vaginal administration can be presented as pessaries, tampons, creams, gels, pastes, foams, or spray formulas containing, in addition to the active ingredient, such carriers as are known in the art to be appropriate.

One of ordinary skill in the art will readily appreciate that the agent that inhibits Cripto-1 of the present inventive methods can be modified in any number of ways, such that the therapeutic efficacy of the agent is increased through the modification. For instance, the agent that inhibits Cripto-1 could be conjugated either directly or indirectly through a linker to a targeting moiety. The practice of conjugating agents to targeting moieties is known in the art. See, for instance, Wadwa et al., J. Drug Targeting 3: 111 (1995), and U.S. Pat. No. 5,087,616. The term “targeting moiety” as used herein, refers to any molecule or agent that specifically recognizes and binds to a cell-surface receptor, such that the targeting moiety directs the delivery of the agent to a population of cells on which surface the receptor is expressed. Targeting moieties include, but are not limited to, antibodies, or fragments thereof, peptides, hormones, growth factors, cytokines, and any other naturally- or non-naturally-existing ligands, which bind to cell surface receptors; The term “linker” as used herein, refers to any agent or molecule that bridges the agent that inhibits Cripto-1 to the targeting moiety. One of ordinary skill in the art recognizes that sites on the agent that inhibits Cripto-1, which are not necessary for the finction of the agent, are ideal sites for attaching a linker and/or a targeting moiety, provided that the linker and/or targeting moiety, once attached to the agent that inhibits Cripto-1; do(es) not interfere with the function of the agent, i.e., the ability to inhibit the Cripto-1 protein.

Alternatively, the agent that inhibits Cripto-1 can be modified into a depot form, such that the manner in which the agent that inhibits Cripto-1 is released into the body to which it is administered is controlled with respect to time and location within the body (see, for example, U.S. Pat. No. 4,450,150). Depot forms of agents can be, for example, an implantable composition comprising the agent that inhibits Cripto-1 and a porous material, such as a polymer, wherein the agent is encapsulated by or diffused throughout the porous material. The depot is then implanted into the desired location within the body and the agent that inhibits Cripto-1 is released from the implant at a predetermined rate by diffusing through the porous material.

Furthermore, the present inventive method can comprise the administration of the agent that inhibits Cripto-1 with an agent that enhances its efficacy. The agent that inhibits Cripto-1 and the agent that enhances it efficacy can be administered simultaneously or sequentially, by the same route or a different route.

For purposes of all of the present inventive methods, the amount or dose of the agent administered should be sufficient to effect a therapeutic response in the animal over a reasonable time frame. Particularly, the dose of the agent that inhibits Cripto-1 should be sufficient to inhibit the Cripto-1 protein in a cell within about 1-2 hours, if not 3-4 hours, from the time of administration. The dose will be determined by the efficacy of the particular agent and the condition of the animal (e.g., human), as well as the body weight of the animal (e.g., human) to be treated. Many assays for determining an administered dose are known in the art. For purposes of the present invention, an assay, which comprises comparing the extent to which the protein is inhibited in a cell upon administration of a given dose of an agent to a mammal among a set of mammals of which is each given a different dose of the agent, can be used to determine a starting dose to be administered to a mammal. The extent to which the Cripto-1 protein is inhibited upon administration of a certain dose can be assayed by a SMAD-luciferase assay (see Bianco et al, Mol. Cell. Bio. 22: 2586-2597 (2002) and Bianco et al., Cancer Res. 63: 1192-1197 (2003)).

The dose also will be determined by the existence, nature and extent of any adverse side effects that might accompany the administration of a particular agent that inhibits Cripto-1. Ultimately, the attending physician will decide the dosage of the agent that inhibits Cripto-1 with which to treat each individual patient, taking into consideration a variety of factors, such as age, body weight, general health, diet, sex, inhibitor to be administered, route of administration, and the severity of the condition being treated.

With respect to the present inventive method of inhibiting progression of a neurodegenerative disease, the neurodegenerative disease can be any of those discussed herein. Preferably, the neurodegenerative disease is NeuroAIDS, Alzheimer's disease, multiple sclerosis, ALS, Parkinson's disease, or encephalitis.

Furthermore, the mammal can be any mammal as discussed herein. Preferably, the mammal is a human.

### EXAMPLES

### Abbreviations

For convenience, the following abbreviations are used herein:

Human immunodeficiency virus (HIV-1); central nervous system (CNS); Human immunodeficiency virus-encephalitis (HIV-E); acquired immunodeficiency syndrome (AIDS); simian immunodeficiency virus (SIV); simian immunodeficiency virus-encephalitis (SIV-E); cerebrospinal fluid (CSF); simian human immunodeficiency virus (SHIV); ainyotrophic lateral sclerosis (ALS); Teratocarcinoma-derived Growth Factor-1 (TDGF-1); Epidermal Growth Factor-cysteine rich motif (EGF-CFC); National Center for Biotechnology Information (NCBI); Polymerase Chain Reaction (PCR); ligase chain reaction (LCR); strand displacement amplification (SDA); repair chain reaction (RCR); cyclic probe reaction (CPR); Reverse transcription-PCR (RT-PCR); glial fibrillary acidic protein (GFAP); Moloney murine leukemia virus (MMLV); sodium dodecyl sulfate (SDS); interleukin-6 (IL6); interferon (UN); N-methyl-D-aspartate (NMDA); nerve growth factor (NGF); brain-derived neurotrophic factor (BDNF); tyrosine kinase (TrK); ciliary neurotropic factor (CNTF); leukemia inhibitory factor receptor (LIFR-P); corticotrophin releasing factor receptor 1 (CRFR1); corticotrophin releasing factor (CRF); phosphate-buffered saline (PBS); epidermal growth factor (EGF); enzyme linked immunosorbent assay (ELISA); leukocyte interferon inducible peptide (LIIF); Transforming growth factor (TGF); and Heparin-binding-epidermal growth factor (HB-EGF); 3,3′-diaminobenzidine (DAB).

The following examples further illustrate the invention but, of course, should not be construed as in any way limiting its scope.

### Example 1

This example demonstrates the histopathology in macaques with SHIV_{500LNV} for two weeks.

Macaque AX62 was an uninfected age-matched pig-tailed macaque, which exhibited no obvious signs of neurological dysfunction and exhibited no neuropathology at necropsy. The other four pig-tailed macaques used in this study were AX67, CM6G, CB4R, and CBRW (Singh et al., Virology 296: 39-51 (2002)). These macaques were inoculated with pathogenic SHIV_{500LNV}, and virus loads were followed for two weeks prior to euthanasia (Singh et al. (2002), supra). All four of these macaques developed astrocytosis, some developed a transient meningitis, and all had extensive neuroinvasion by the virus (Singh et al. (2002), supra; Table 1).

At the time of euthanasia, animals were anesthetized by administration of 10 mg/kg ketamine intramuscularly, followed by sodium pentobarbital at 20-30 mg/kg intravenously. A laparptomy was performed, and the animal was exsanguinated by aortic canulation. The left ventricle was canulated, the right atrium was nicked, and the animal was perfused with one liter of cold pyrogen-free Ringer's saline. The left half of the brains from infected and uninfected control macaques were fixed by immersion in 2% paraformaldehyde. Regions were blocked in a standard coronal plane into 6-mm blocks, cryoprotected in 30% sucrose in 0.1 M phosphate buffer, and frozen-sectioned at 50 μm using a sliding microtome. For histopathology, the right half of the brains were fixed by immersion in 10% neutral buffered formalin, and blocks containing frontal, motor, parietal, occipital, and temporal cortex, corpus callosum, basal ganglia, thalamus, midbrain, pons, medulla and cerebellum, and cervical, thoracic and lumbar spinal cord were embedded in paraffin and sectioned at 5 μm. Sections were stained with hematoxylin and eosin for routine histopathological analysis.

In this study, the histological lesions, as well as levels of astrocyte, in macaques inoculated with SHIV_{500LNV} were examined. The results of the immunohistochemical staining of brain sections for glial fibrillary acidic protein (GFAP) from a control macaque demonstrated that there was minimal staining of the pia and little or no staining of perivascular astrocytes. In contrast, the immunohistochemical staining of brain sections for GFAP from macaque CM6G (inoculated with SHIV_{500LNV}) revealed intense GFAP staining of astrocytes lining the pia and blood vessels of the gray and white matter. En addition to astrocyte activation, meningitis was observed at frontal, motor, parietal, occipital and temporal cortices and spinal cord. Occasional small microglial nodules were observed in the spinal cord. The levels of astrocyte activation in the macaques in the study are summarized in Table 1.

TABLE 1
Macaques analyzed in this study, presence of viral sequences
in different regions of the brain, and level of astrocytosis.

| | | | ^{a}Number of | |
|---|---|---|---|---|
| | | | regions of | |
| | Virus | | brain positive | |
| | inoculated | Duration | for viral | |
| Macaque | infection | of | sequences | ^{b}Astrocytosis |
| AX62 | None | N/A | None | None |
| CM6G | SHIV_{500LNV} | 2 weeks | 13/14 | ++++ |
| AX67 | SHIV_{500LNV} | 2 weeks | 15/15 | ++++ |
| CB4R | SHIV_{500LNV} | 2 weeks | 13/15 | ++++ |
| CB4W | SHTV_{500LVN} | 2 weeks | 13/15 | ++++ |

^{a}Determined by DNA PCR.
^{b}++++ = intense astrocyte activation.

The astrocyte activation and histopathology were comparable to that observed in short-term infections of macaques that were inoculated with SIV_{mac} (Berman et al., Mol. Chem. Neuropathol. 34: 25-38 (1998); Berman et al., Neurobiol Dis. 6: 486-498 (1999); and Raghavan et al., Neuropathol. Appl. Neurobiol. 25: 285-294 (1999)) and humans that have developed neuroAIDS (Rappaport et al., J. Leuk. Biol. 65: 458-465 (1999); Vitkovic, Curr. Top. Microbiol. Immuunol. 202: 105-116 (1995); and Wesselingh et al., Curr. Opin. Neural. 14: 375-379 (2001)).

This example demonstrated that a strong host response was associated with SHV neuroinvasion.

### Example 2

This example demonstrates a cDNA analysis of immunomodulatory gene expression of cortical regions from a normal macaque versus one with neuroAIDS.

In order to identify genes that were differentially expressed in SHIV infected and uninoculated macaques, cDNA array analysis was performed using the human cytokine cDNA array from Clontech (catalog #7744-1). At necropsy, the CNS was dissected into 14 different regions, and tissues were frozen in liquid nitrogen and stored at −85° C. until used for RNA extractions. RNA was prepared from the parietal cortex tissue by homogenization in the TRIZOL reagent (InVitrogen,Carlsbad, Calif.), and total RNA was isolated as per the manufacturer's instructions. The poly (A)-enriched RNA fraction was isolated using the Atlas Pure RNA isolation kit (Clontech) and was used directly to generate cDNA probes using Moloney murine leukemia virus (MMLV) reverse transcriptase, ³²P-dATP, and random Atlas array specific primers. Probes were gel-purified by NucleoSpin Extraction Spin columns, and used to hybridize to nylon arrays overnight at 68° C. Nylon filters were washed a total of five times (first three washes in 2×SSC, 1% sodium dodecyl sulfate (SDS) for 30 minutes at 68° C.; the fourth wash in 0.1×SSC with 0.5% SDS for 30 minutes at 68° C.; and the final wash in 0.1×SSC, 0.5% SDS for 5 minutes at room temperature). Membranes were analyzed on a Packard Cyclone phosphoimaging system at a resolution of 50 μm. The spot intensities were measured with Atlaslmage 2.0 software. As macaques are an outbred species and thus subject to more animal to animal variation in gene expression, a more stringent 2.5 difference was chosen as the arbitrary cutoff value for significant difference in gene expression. Relative levels of gene expression between arrays were calculated by dividing the normalized intensities of spots on one array by normalized spot intensities on a second array using user-defined comparison with standard housekeeping genes.

The results of cytokine cDNA array analysis were confirmed by performing RT-PCR with oligonucleotide specific for three genes from the cDNA array that showed a 2.5-fold or greater increase or decrease compared to the normal control macaque AX62. Three genes were teratocarcinoma-derived growth factor (TDGF or Cripto), CD40 antigen and interleukin-6 (IL6). The oligonucleotides used in the RT-PCR amplification were based on human sequences in the Genbank: TDGF: 5′-AAGCTATGGACTGCAGGAAGATGG-3′ (sense; SEQ ID NO: 3) and 5′-AGAAAGGCAGATGCCAACTAGC-3¹ (antisense; SEQ ID NO: 4); IL-6: 5′-CGCCTTCGGTCCAGTTGCCTTCT-3′ (sense; SEQ ID NO: 5) and 5′-ATCCAGATTCCAAGCATCCATC-3′ (antisense; SEQ ID NO: 6); and LIIF: 5′-ATGCGCCAGAAGGCGGTATCCG-3′ (sense; SEQ ID NO: 7) and 5′-CTACTCCTCATCCTCCTCACTATC-3′ (antisense; SEQ ID NO: 8). The RT-PCR was performed with equal amounts of total RNA and the Titan One-Tube RT-PCR System (Roche Diagnostics, Indianapolis, Ind.) using an initial denaturation step at 94° C. for 2 minutes. This was followed by 10 cycles with denaturation at 94° C. for 30 seconds, annealing at 55° C. for 30 seconds, and elongation at 68° C. for 45 seconds. This was followed by 25 cycles with denaturation at 94° C. for 30 seconds, annealing at 55° C. for 30 seconds, and elongation at 68° C. for 2 minutes. At the end of the above cycling profile, a 10-minute elongation step was performed at 68° C. Following the PCR amplification, a 10 μl aliquot was removed and run on a 1.5% agarose gel, and bands were visualized by staining with ethidium bromide.

These studies were aimed at examining which cytokines were elevated in the CNS in SHIV encephalitis. Using immunomodulatory cDNA arrays, cortical tissue from normal uninfected macaque (AX62) and from macaques inoculated with pathogenic SHIV_{500LNV} were examined for two weeks. Shown in Table 2 are the immunomodulatory genes whose expression was up-regulated more than 2.5 fold in all four macaques (8 genes) when compared to the uninfected control macaque (AX62).

To confirm the up-regulation of genes identified in the cDNA arrays (Table 1), oligonucleotides based on the human sequences of Cripto-1, LIIF, and IL-6 were used in RT-PCR of parietal cortex RNA samples. The mRNA expression of all three genes was clearly upregulated in macaque AX67, thus confirming the results of the cDNA array (Table 2). Similar results were obtained for the other SHIV_{500LNV}-inoculated macaques.

TABLE 2
Cytokine array genes upregulated >2.5 fold in all four
macaques compared to uninfected, age-matched macaque.

| | Average difference in | Accession |
|---|---|---|
| Gene or product | expression (fold) | No. |
| Leukocyte interferon inducible | 30.09 | X02492 |
| peptide | | |
| Corticotropin releasing factor | 20.68 | X72304 |
| receptor 1 precursor (CRF-R; | | |
| CRF1) | | |
| Interleukin-6 precursor | 15.03 | M1 45 84 |
| Teratocarcinoma -derived growth | 9.56 | M96955 |
| factor | | |
| CDW40 antigen | 9.12 | X60592 |
| Cysteine-rich fibroblast growth | 6.02 | U64791 |
| factor | | |
| Neurotrophin-3 precursor | 4.84 | X52946 |
| Ciliary neurotrophic factor | 4.74 | M73238 |
| receptor | | |

These results indicate that several genes were dysregulated in the cortex of all four macaques when compared to the uninoculated, age-matched control macaque. The genes whose expression was up-regulated fall into three broad categories: genes upregulated during the early inflammatory response to viral infection, genes upregulated during the host neuroprotective response, and genes with unknown function in the nervous system.

The first category includes those molecules that were up-regulated as a consequence of the virus infection and the early inflammatory response. These include LIIF, CD40W antigen, and the cysteine-rich fibroblast growth factor receptor. The highest level of up-regulation observed was LIIF or 6-16 (Friedman et al., Cell 38: 745-755 (1984)). Expression of 6-16 is selectively stimulated by interferon-α (Ackrill et al., Nucleic Acids Res. 19: 591-598 (1991)) as part of the interferon antiviral response (Grandvaux et al., Curr. Opin. Infect. Dis. 15: 259-267 (2002)). CDW40 antigen (nerve growth factor receptor-related B-lymphocyte activation molecule, tumor necrosis factor receptor superfamily member 5 precursor) was first described as a surface molecule present on B cells and carcinomas induced by interferon gamma. This molecule is a member of the tumor necrosis factor receptor superfamily, is up-regulated by interferon (IFN)-gamma and is engaged by CD40L, and found on CD4⁺T cells, B cells, monocytes, and microglia. Thus, the CD40-CD40L interactions may be important in central nervous system inflammatory diseases, such as HIV-1 encephalitis. A recent study showed that the number of CD40 positive microglia was increased in the brains of people with HIV-1 encephalitis (D'Aversa et al., Am. J Pathol. 160: 559-567 (2002)).

Cysteine-rich fibroblast growth factor receptor (also known as Golgi membrane sialoglycoprotein) is localized in Golgi cistemae (Kawano et al., Histochem Cell Biol. 17(5):381-9 (2002)). Antibodies directed against MB 160 have been used to identify changes in Golgi apparatus in neurodegenerative disorders, such as Creutzfeld-Jacob disease (Sakurai et al., Acta Neuropathol. (Berl), 100: 270-274 (2000)). This protein is found in subependymal astrocytic processes and perivascular astrocytic end feet (Gonatas et al., Brain Res. 855: 23-31 (2000)). Increases during inflammation, combined with potential expression by activated astrocytes, suggests that the protein may be expressed by subpial astrocytes, which. are highly activated at this early stage of the disease in these macaques (Singh et al., Virology296: 39-51 (2002)).

A number of the genes that were up-regulated (IL-6, CFTR, NT-3, and CRFR) have been associated with inflammatory and/or neuroprotective fimctions and may represent the host brain response to early neuroinvasion by the virus. IL-6 is made by activated astrocytes after brain injury. HIV Tat induces-IL-6 in astrocytes (Nath et al., J. Biol. Chem. 274: 17098-17102 (1999)) and brain endothelial cells (Zidovetzzi et al., AIDS Res. Hum. Retro. 14: 825-833 (1998)). IL-6 is neuroprotective in the NMDA excitotoxicity model (Toulnond et al., Neurosci Lett. 144: 49-52 (1992)). Over-expression of IL-6 using the GFAP promoter improves healing following cortical injury and revascularization of injured neural tissue (Swartz et al., Brain Res. 896: 86-95 (2001)). IL-6-deficient mice have a slower rate of healing following injury to the cerebral cortex, and the blood brain barrier is leaky in IL-6 knockout mice following cortical injury. Following cortical freeze lesions, injury responses, such as expression of metallothionein I and II, are reduced in IL-6 knockout mice. The IL-6 KO mice also showed higher levels of inducible nitric oxide synthase, also suggesting that IL6 expression after injury is neuroprotective (Penkowa et al., Glia 32: 271-285 (2000)). Increased expression of IL-6 two weeks after inoculation with SHIV_{500LNV} is most likely a neuroprotective response.

NT-3 is a member of the neurotrophin family that also includes NGF, BDNF, and NT-⅘. The neurotrophins act through tyrosine kinase (TrK) receptors to promote neuronal survival. Increases in NT-3 expression after viral inoculation have not been described previously. In contrast, Boma virus has been shown to reduce the expression of NT-3 two weeks postinoculation in the rat hippocampus, and Borna virus infection is associated with loss of neurons in the hippocampal dentate gyrms (Zocher et al., J. Neurovirol. 6: 462-477 (2000)).

Expression of CNTFR the receptor for CNTF, was also upregulated in the SHIV inoculated macaques. CNTF is a multifunctional growth factor found in the central nervous system that omots-neuron survival after injury (Oliveira et al., J. Comp. Neurol. 447: 381-393 (2002)). CNTFR is the ligand-binding component of the CNTF receptor, which is associated with two signaling components, gp130 and LIFR-P. CNTF binds to CNTFR-α, allowing recruitment of gp130 and LIFR-P to form a tripartite receptor complex. CNTFR is expressed by neurons, and expression increases after injury (D)uberley et al., Neurosci. Lett. 218: 188-192 (1996)). CNTF knockout mice develop normally, while mice lacling CNTFR-α die perinatally and have severe motor neuron deficits, suggesting that there are additional ligands for CNTFR (DeChiara et al., Cell 83: 313-322 (1995)), including cardiotrophin-like cytoline (Elson et al., Nat. Neurosci. 3: 867-872(2000)). CNTFR-α has been shown to induce secretion of cardiotrophin-like cytoline and to mediate its functional responses (Plun-Favreau et al., EMBO J. 20: 1692-1703 (2001)). This cytokine, also known as neurotrophin-1/B cell-stimulating factor-3, stimulates B cell function and antibody production (Senaldi et al., J. Immunol. 168: 5690-5698 (2002)). Thus, increased expression of CNTFR may be simultaneously involved in neuroprotection and immune responses to virus.

CRFR1 is the high afnity receptor for corticotrophin releasing factor. It is a seven-transmembrane domain G-protein coupled receptor. CRFR1 has been localized in the macaque brain, and is found throughout the neocortex in all layers (Sanchez et al., J. Comp. Neurol. 408: 365-377 (1999)). This receptor is thought to mediate effects of CRF on affective regulation and cognitive function (Sanchez et al., J. Comp. Neurol. 408: 365-377 (1999)). CRF is involved in the acute phase and the recovery phase of the stress response (Real et al., Curr. Opin. Pharmacol. 2: 23-33 (2002)), and HIV gp120 has been shown to stimulate expression of CRF mRNA in rat hypothalamic tissue (Pozzoli et al., J. Neuroimmunol. 118: 268-276 (2001)).

The last category of genes has an unknown function(s) in the CNS and includes the gene for Cripto. Since this molecule has not been described in great detail in the central nervous system, further characterization of the cell type in which this molecule was expressed was deemed necessary. Cripto is a member of the EGF-CFC family, including mouse Cripto (Dono et al., Development 118: 1157-1168 (1993)), chicken Cripto (Colas et al., Gene 255: 205-217 (2000)), Xenopus FRL1 (Kinoshita et al., Cell 83: 621-630 (1995)), mouse Cryptic (Shen et al., Development 124: 429-442 (1997)), and zebrafish Oep (one-eye pinhead) (Zhang et al., Cell 92: 241-251 (1998)). The functions of Cripto have been more widely studied in cancer cells. Expression of cripto is upregulated in human colon, gastric, pancreatic, lung and breast carcinomas (Salomon et al., Endocrine-Related Can. 7: 199-226 (2000)). Cripto is thought to be involved in cell transformation because increases in cripto expression can be detected in early, premalignant lesions (Niemeyer et al., Int. J. Cancer 81: 588-591 (1999)). Transfection of Cripto-1 into mammary epithelial cells enhances growth in soft agar and in serum-free medium, increases proliferation, increases formation of branching, duct-like structures, and increases cell migration (Wechselberger et al., Exp. Cell. Res. 266: 95-105 (2001)). Cripto acts as a survival factor in mouse mammary epithelial cells and human cervical carcinoma cells, when they are grown in low serum medium (Ebert et al., Exp. Cell Res. 257: 223-229 (2000); and Niemeyer et al., Cell Death Differ. 5: 440-449 (1998)). Cripto enhances the tyrosine phosphorylation of Erb B-4, an oncogenic receptor tyrosine kinase involved in breast cancer (Bianco et al., J. Biol. Chem. 274: 8624-8629 (1999)). Expression of Cripto is also critical in early embryogenesis and brain development, and Cripto regulates growth of tumor cells (Ding et al., Nature 395: 702-707 (1998); Xu et al., Developnment 126: 483-494 (1999); and Zhang et al., Cell 92: 241-251 (1998)). Cripto is expressed in the entire embryonic ectoderm at the time of implantation (Johnson et al., Dev. Dyn. 201: 216-226 (1994)). During embryogenesis, Cripto is involved in the specification of the primitive streak, embryonic mesoderm and endoderm, and in positioning of the anterior-posterior axis. Expression of Cripto protein allows cells to respond to instructive Nodal signals (Gritsman et al., Cell 97: 121-132 (1999)). These instructive Nodal signals are involved in regional specification of the ventral telencephalon and forebrain and specification of brain left-right asymmetries (Concha et al., Neuron 28: 399-409 (2000)). Embryos lacking Cripto die in utero. Embyros do not undergo gastrulation and formation of germ layer resulting in the absence of the primitive streak (Ding et al., Nature 395: 702-707 (1998); and Zhang et al., Cell 92: 241-251 (1998)). While a previous study identified expression of Cripto mRNA in the adult mouse brain, the specific cell types and regional localization were not examined (Dono et al., Development 118: 1157-1168(1993)). The results of this study identify widespread expression of Cripto in neurons, with both perikaryal cytoplasmic and dendritic localization. The function of Cripto in neurons of the adult brain and its up-regulation in the brains of a SHIV-infected macaques are also unknown. By analogy with its known function in tumor cells and the localization of Cripto in dendrites, Cripto may have a role in the formation and/or maintenance and branching of dendrites and may actually be neuroprotective. Whether Cripto expression is elevated during the course of neuroAIDS due to the enhanced expression of one or more cytokines/chemokines remains to be determined.

This example demonstrated the upregulation of several distinct genes in neuroAIDS.

### Example 3

This example demonstrates that macaque Cripto-1 is localized to neurons throughout the CNS.

Microglial and astrocyte activation were assessed using immunohistochemistry to visualize MHC-II and GFAP. Blocks were cryoprotected in 30% sucrose in 0.1 M phosphate-buffer and were frozen-sectioned at 50 μm using a sliding microtome. Sections were incubated free-floating in pre-block solution (10% normal goat serum in phosphate-buffered saline (PBS)), washed, and incubated in primary antibody overnight at room temperature. Sections were washed, incubated in biotinylated goat anti-mouse or anti-rabbit IgG diluted 1:100, washed and incubated according to the protocol supplied by Vector Laboratories, Burlingame, Calif. in their ABC Elite kit, and finally washed and reacted with 0.5% diaminobenzidine with 0.1% H₂O₂. The primary antibodies used were mouse monoclonal anti-MHC-H (LN-3, ICN Biomedical, Cosa Mesa, Calif.) diluted 1:200 and mouse monoclonal anti-GFAP (Boehringer-Mannheim Biochemicals, Indianapolis, Ind.) diluted 1:100. Controls consisted of incubating the sections with buffer in the place of the primary antibody. For visualization of Cripto by immunohistochemistry, a rabbit polyclonal antibody (#1579) generated against a 17-mer peptide corresponding in sequence to the last 17 amino acids in the epidermal growth factor (EGF)-like domain of the human CR-1 protein was used. This rabbit antibody recognizes full-length recombinant CR-1 protein (˜28 kDa) by Western blotting and does not cross-react with any other EGF-like peptide in an enzyme linked immunosorbent assay (ELISA), such as EGF, TGF alpha, amphiregulin, HB-EGF or heregulin beta-1. The reactivity of this antibody is similar to the CR67 antibody previously described (Qi et al., Br. J. Cancer 69: 903-910 (1994)).

To visualize Cripto in the CNS, 50 μm frozen sections were washed free floating (1× in PBS, pH 7.5) and eqhilibrated in PBS, quenched for endogenous peroxidase (in 0.6% H₂O₂ for 30 minutes), and blocked in 10% normal goat serum (in PBS) for one hour. Sections were incubated in primary antibody (1:1,000) overnight at room temperature, washed three times in PBS, and incubated in biotinylated goat anti-rabbit IgG (1:200) for 1 hour. After ABC steps, sections were washed three times in PBS and rinsed in 0.5% Triton X-100 for 30 seconds and incubated with 3,3′-DAB for 2-10 minutes until suitable color development. One set of controls for non-specific staining consisted of incubation of the sections with buffer in the place of normal rabbit serum. Sections were rinsed in distilled water, mounted on gelatin coated glass slides, dried over night at room temperature, dehydrated through alcohol and xylene and covered with cover slips as per routine histological procedures. Experiments in which the rabbit polyclonal antibody was pre-incubated with the 17-mer peptide used to immunize the rabbits were also performed. This blocking peptide was incubated with the antibody (1:1,000 dilution) at a concentration of 50 μg/ml overnight at 4° C. prior to use in immunohistochemistry. The absence of staining in neurons indicated that Cripto antibodies used in the staining are indeed specific to the antigen.

To determine the cell types in the CNS that expressed the Cripto, immunohistochemical experiments were performed using a rabbit polyclonal antibody generated against a Cripto-1 specific epitope (Qi et al. (1994), supra). Immunoreactivity was present in neurons throughout the cerebral cortex. The staining was densest in pyramidal neurons of layers II-III, V and VL but additional non-pyramidal neurons in the deep aspect of layer VI were also stained. In other regions of the brain, Cripto immunoreactivity was present in neurons of the dentate and interpositus nuclei of the cerebellum and in neuropil of the molecular and granule layer of the cerebellar cortex. Widespread staining was also present in neurons in the basal ganglia, brainstem and thalamus, notably in neurons of the lateral geniculate nucleus and other thalamic nuclei, globus pallidus, sustantia nigra pars compacta, thalamic reticular nucleus, hippocampal pyramidal and neurons-of the hippocampal molecular layer. The immunostaining of neurons was specific as pre-incubation of the polyclonal antibody with the peptide used to make the antiserum abolished neuronal staining.

This example demonstrated that Cripto-1 is localized to neurons throughout the CNS.

### Example 4

This example demonstrates that Cripto mRNA is ,observed in multiple regions of the CNS.

As Cripto protein was detected in the neurons of the cerebral cortex, it was determined whether the expression of Cripto RNA was widespread in the CNS or if it was localized to select regions. RNA was extracted from 10-15 regions of the CNS from four inoculated macaques and control macaque AX62. The RNA was used in RT-PCR using oligonucleotide primers specific for Cripto. The majority of the regions analyzed were positive for Cripto (see Table 3).

TABLE 3

| SHIV uninoculated | | SHIV inoculated | | | |
|---|---|---|---|---|---|
| Region tested | AX62 | AX67 | CB4R | CM6G | CB4W |
| FC | − | + | + | + | + |
| PC | + | + | + | − | + |
| TC | + | + | + | + | + |
| CC | + | + | + | + | − |
| BG | + | − | + | − | + |
| MB | + | + | + | + | + |
| PN | + | + | + | + | + |
| MD | − | + | + | + | + |
| CB | + | + | ND | + | + |
| CSC | + | + | + | + | + |
| TSC | + | + | + | + | + |
| LSC | + | − | + | − | − |
| CR | ND | + | ND | ND | ND |
| MC | ND | + | + | + | + |
| OC | ND | + | + | − | + |
| HIP | ND | + | + | ND | + |
| LN | ND | − | + | ND | − |
| TH | ND | ND | ND | − | ND |

+: positive for Cripto; −: negative for Cripto; ND: no data; FC: frontal cortex; PC: parietal cortex; MC; motor cortex; OC: occipital cortex; TC: temporal cortex; BG: basal ganglia; HIP: hippocampus; TH: thalamus; MD: midbrain; PN: pons; MED: medulla; CB: cerebellum; CSC: cervical spinal cord; TSC: thoracic spinal cord; LSC: lumbar spinal cord; LN: lymph node.

Although the macaques were exsanguinated and perfused with saline at necropsy, which removes the majority of the blood and, thus, possible contamination of the CNS, several controls were included to rule out possible blood contamination. First, RNA was extracted from 1×10⁶ isolated PBMC, which is roughly equivalent to the number of PBMC in approximately 1 ml of blood (and greater than the volume of tissue from which RNA was extracted). RT-PCR performed using the oligonucleotide primers to Cripto failed to amplify a product.

This example demonstrated the tissue distribution of Cripto mRNA.

### Example 5

This example demonstrates a method of detecting the expression of Cripto-1 in human patients with multiple sclerosis (MS), amyotrophic lateral sclerosis (ALS), Parkinson's disease, Alzheimer's disease, or encephalitis.

Brain tissue and cerebrospinal fluid will be taken from patients suffering from MS, ALS, Parkinson's disease, Alzheimer's disease, or encephalitis. Cripto-1 expression will be detected using immunohistochemical methods described in Adkins et al., J. Clin. Invest. 112: 575- 587 (2003) using an anti-Cripto-1 antibody also described therein. Brain tissue samples obtained from the patients will be analyzed for Cripto-1 expression using double sandwich ELISA methods described in Bianco et al., J. Cell. Physiology 190(1): 74-82 (2002).

The expression of Cripto-1 may correlate with the neurodegenerative diseases and could be used as a marker for detecting the diseases. Agents that inhibit Cripto-1, i.e., Cripto-1 inhibitors, could be used to inhibit the progression of the neurodegenerative disease.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of detecting a neurodegenerative disease in a mammal, which method comprises assaying the copy number of a Cripto-1 gene or the expression level of a Cripto-1 gene product in the central nervous system of the mammal, wherein an amplification of the Cripto-1 gene or an overexpression of the Cripto-1 gene product is indicative of a neurodegenerative disease in the mammal.

2. The method of claim 1, wherein the neurodegenerative disease is selected from the group consisting of NeuroAlDS, Alzheimer's disease, multiple sclerosis, amyotrophic lateral sclerosis (ALS), Parkinson's disease, and encephalitis.

3. The method of claim 1, wherein the mammal is a human.

4. The method of claim 1, wherein the method comprises using a cDNA array and/or comprises non-quantitative reverse transcription-polymerase chain reaction (RT-PCR).

5. The method of claim 4, wherein the RT-PCR is carried out with oligonucleotide probes consisting essentially of the nucleotide sequences AAGCTATGGACTGCAGGAAGATGG (SEQ ID NO: 3) and AGAAAGGCAGATGCCAACTAGC (SEQ ID NO: 4).

6. The method of claim 1, wherein the expression level of a Cripto-1 gene product is assayed from cerebrospinal fluid obtained from the mammal.

7. A method of inhibiting progression of a neurodegenerative disease in a mammal, which method comprises administering to the mammal an agent that inhibits Cripto-1 in an amount effective to inhibit Cripto-1 in the central nervous system of the mammal, whereupon the progression of the neurodegenerative disease is inhibited.

8. The method of claim 7, wherein the neurodegenerative disease is selected from the group consisting of NeuroAIDS, Alzheimer's disease, multiple sclerosis, ALS, Parkinson's disease, and encephalitis.

9. The method of claim 7, wherein the mammal is a human.

10. The method of claim 7, wherein the agent is an oligonucleotide that hybridizes to a nucleic acid molecule encoding a Cripto-1 protein.

11. The method of claim 7, wherein the agent is an antibody that specifically binds to a Cripto-1 protein.

12. The method of claim 7, wherein the agent is a peptide that specifically binds to a Cripto-1 protein.

13. The method of claim 7, wherein the agent is a mutant Cripto-1 protein.

14. An isolated or purified oligonucleotide consisting essentially of the sequence of AAGCTATGGACTGCAGGAAGATGG (SEQ ID NO: 3) or AGAAAGGCAGATGCCAACTAGC (SEQ ID NO: 4).


SEQUENCE LISTING (10 sequences)

SEQ ID NO: 1 - DNA, 7355 bp, Homo sapiens
  misc_feature (3154)..(3154): n is a, c, g, or t
ctgcagtcagtacctatggggaatgggaatctcttcagggggttctggtccttagagaagggattttgtgtggctgaaaagctggcatcctggggttctggtcccgtcaatgacaacatcgccagactggagacctcagttacttcctctgaaaatgcagtgatttccaggggtcctatttaagcctctaaaaattccacaagagctttagataaggaaatagcaacgcaggggtgtgttcttttgccagttctgccacaggctgcccagtctctaaagacaagacatccaaatcccccaatagaactagttgtcttgtccataaagtgagactaatattgtgggcgctacttatctacttagcaccttggactggtgaggactgtggtgcacaagctaccttacaaatgtaccacactgagtaaccatctttaaaccttcctttgcagctccagggctagccttctcctttgcgagccctccccacctcggcctcctagagcttcaggccatgttcccctgtccctgtgaatctcagcatgctacctgaagcatttcacctgaaaaggccacacagggaggaggcgaagcgcagcaggaatgaaatagtcaactgctgtggagttggaaatgttgctgcatcccaccattgactggatggggccctcactcccccaatacaaattattttacgtttgcttctcccaagatcatgtgtaaggccgggcgcggtagctcatgcctgtaatcccagcactttgggaggccgaggcgggtggatcatgaggtcaggaatttgagaccagactgaccaacatggtgaaaccctgtctctactgaaaatacaaaaattagccgggcgttgtgcgggcgcctgtaatcccagctactcaggaggttgaggcaggagaatgaggcaggagaatcacttgaacccaggaggtggaggttgcagtgagccaagatcgtgccattgcactccagcccgggtaacagagggagactctgtcgcaaaaaaaaaaaaaaaaaaaaaaaaaaaatcatgtgtagaagtaaataacacctcttcccagcacctgtggatccatggcattcacacaggtaggcactgacgctgaaatggcctcagatgctaccaattctttctgccgaggcctaaatccaataaacagagacaactatctaataaagtttcgcattgtgtgcctggcaacattaagtaaccgtcaggctttcctttagaaattggaattaaatgcgatttagaaactattaacgactttgggcgtatttaataacacatgaaataacccggaggattgaaatgttaggtgaggcgactccggctcatagacgcgcctctccatctggggcgtctggcacttagtggaaccactcaataaacacgtttacccctgcaagcggcacatcagagtccgggggtaattctcggtgtcgtggggccaggacggcgaggggctggaagaggccgccctgtgggagctgggaggctgagataaattcccgtgattgggtgctgaaatggcctcccatgccggactgccgtggttctagaactttttcctggaacaggccggcactcccactggagagtcccagctgcctctggccgcccctcccctctcccgggcacctggcgccgctcccgcgtcctttcaggaattcacgtccgcctggaatttgcacttcaagtctggagcccccaaggaacccctcctgaccctgaacttctatctcagtttcaagcttcctagtcttccccacacacacacacctagctcctcaggcggagagcacccctttcttggccacccgggtatcccccagggagtacggggctcaaaacacccttctggaaaaaacaaaggtggaagcaaatttcaggaagtaaaacttctgaaataaaataaaatatcgaatgccttgagacccatacattttcaggttttcctaattaaagcaattactttccaccacccctccaacctggaatcaccaacttgattagagaaactgatttttcttttttctttttttttcccgaaaagagtacctctgatcattttagcctgcaactaatgatagagatattagggctagttaaccacagttttacaagactcctcttcccgcgtgtgggccattgtcatgctgtcggtcccgcccacctgaaaggtctccccgccccgactggggtttgttgttgaagaaggagaatccccggaaaggctgagtctccagctcaaggtcaaaacgtccaaggccgaaagccctccagtttcccctggacgccttgctcctgcttctgctacgaccttctggggaaaacgaatttctcattttcttcttaaattgccattttcgctttaggagatgaatgttttcctttggctgttttggcaatgactctgaattaaagcgatgctaacgcctcttttccccctaattgttaaaagctatggactgcaggaagatggcccgcttctcttacaggtatgagctaatcttagaatagtgaactttttttgattgctagagattgccagcttaggaagtaatgttctacactgtcatttgatttttctccttgctcaagccttaaaagagctgccaaccgactgctgtttttcctgaaagacctggaatttcacatggttacttctaactttgccattggcttttaacattttcgtgttaatgttaattttcattttatgttaatgactctgcctatgaaatagtgtttctttacttcttgtacaaataaaggtcagtactacaaccaaatttaaatcttccgaaaagattaaaggtataagcagattcaatacttggcaaaactattaagataatagcaaaaaaaaaaaaaaaacccacattttttacctaaaaaccttttaagtgattggttaaaatagtttggccgggtgcggtggctcacgcctgtaatcctagcactttgggaggcagaggcgggtggatcactgaggtcaggagaccagcctggccaacatggcaaaaccccgtctctattaaaaatacaaaaattagccaagcatggtggcgggcacctgtaatcccagctactctggaggctgaggcaggagaattgcttgaactggggaggggaggncagtgagccgagatcgcaccattgcactccagcctgggtgaaaaaccgaaactccctctcaaaaataaataaataaatacagtagtttgtaaaatgattcatcggtaacatgggatgcagctattttttaatccttatatgaaaattgtatgcaggggaaaacatgtgaaatagaagataaaagacatatacctacttaaaattaggtacttatgtgaggacagggcctaagaaataataatatatattaaaaagacttggatattggtgactttttttcaacatttttctttgttacatgaattagccattaaaaaaagaaagatggtgctctacaatttcttttcagtgatctgtggtcttgtccttgtgatgagaggacctgggtgttaacttgtaaggttttatttcctttgtttggctaactcatgtttgacttcctcttcctagtgtgatttggatcatggccatttctaaagtctttgaactgggattagttgccggtgagagaccttttgtttcttttgatcactctcaattttatgtggcctaaaatacagactccatgaattgatttgtcgttaagggctgggccatcaggaatttgctcgtccatctcggggatacctggccttcagagatgacagcatttggccccaggaggagcctgcaattcggcctcggtcttcccagcgtgtgccgcccatggggatacagcacagtaagaactgcctgacttcgatgcttctgccctggcccttcatgtgtctcctgactatctttccaacactctttcacctaaaagggcacctggttctggaactgtgcaggtgctggactgctttggttttggaagtgagacaaggattgtgtattttacttccctagagtgcagtttcctcccctgagtccacttcacactgggaacccagaaccaccactggcctatgcatgaaaatgacttctctgctcaaaggcacagagtcttactctgatacaacacattggtgttgtattaaccttcgcttacaggaattgcccttgcacttttccatccctacacctcagtcattctgttcttacctttcaaggtaaggagctaaacagaacctgctgcctgaatgggggaacctgcatgctggggtccttttgtgcctgccctccctccttctacggacggaactgtgagcacgatgtgcgcaaagagtaagcaattcagaggggcggggagccgtggagaggagagagaaagggaagtggaaatttcagacccaagctatcgcagcttacctgttcattctcaggaactgtgggtctgtgccccatgacacctggctgcccaagaagtgttccctgtgtaaatgctggcacggtcagctccgctgctttcctcaggcatttctacccggctgtggtaagcggaggttctcctctttcttttgccctttgaagttacgtagttgccttggggggtgcttagttagcaggctctccttgtacctcttgtcttgctagagcctggcagccaaagttctgcttataaaagcatcgcagactcctgatgagatagttgccttggcctctttgatatttatttcctcgggaacctggctagtcctgctgcctttcagatagagatgtatttcaagtctatttgacattttatggtctgaacttctattgaggaaaataaacaagtctcggtctcttgttaaaccaagagatgttctctggtgttcctttcctttgggtaggggggacccaaaccaggatgggcagctcatttagagcccaccctgacgacaaattctatcagaggcttggccccttgctagtcctttagaaacttccagagtcctaaaagtccctggtaaccccctccccataccttaccatgactggtcacagaacccttaccatgactggtcacagaaccctttcaccttcttgattttttactgatttgaggaatacaatgaaaagaagggcagcacctggagaggaaaagaggcgacagtcctctctccaccctagcctgagccaggtttctagggccccccaaattcagagacctattatagttctgggccttggagatgtagaaatggaaaatattcaagcccaggaagtaaatgaaagcaaacatttcactgagaacaggaaggaattccccaatccagacagggattgtgtctttgccatttgcatcctgggtgtcaggctcaggataggtgtttgataagtgtgggttgggtgattggatgtgtagggaacatttgctcttcctggaacatggggcccaagtcagaatctaacccaggttgtgctcattcctgcaagtgaaggcatcaccactgggctaggttccaggtgtgagtgtcctgagaagagcaggttcacagtagcgtatagatatgccacatttgtgggcagcaggatgaactgccagagaggtttgctttaatgaccaagcatccctaccttccagatggccttgtgatggatgagcacctcgtggcttccaggactccagaactaccaccgtctgcacgtactaccacttttatgctagttggcatctgcctttctatacaaagctactattaatcgacattgacctatttccagaaatacaattttagatatcatgcaaatttcatgaccagtaaaggctgctgctacaatgtcctaactgaaagatgatcatttgtagttgccttaaaataatgaatacaatttccaaaatggtctctaacatttccttacagaactacttcttacttctttgccctgccctctcccaaaaaactacttcttttttcaaaagaaagtcagccatatctccattgtgcctaagtccagtgtttctttttttttttttttttgagacggagtctcactctgtcacccaggctggactgcaatgacgcgatcttggttcactgcaacctccgcatccggggttcaagccattctcctgcctaagcctcccaagtaactgggattacaggcatgtgtcaccatgcccagctaatttttttgtatttttagtagagatgggggtttcaccatattggccagtctggtctcgaactcctgaccttgtgatccactcgcctcagcctctcgaagtgctgagattacacacgtgagcaactgtgcaaggcctggtgtttcttgatacatgtaattctaccaaggtcttcttaatatgttcttttaaatgattgaattatatgttcagattattggagactaattctaatgtggaccttagaatacagttttgagtagagttgatcaaaatcaattaaaatagtctctttaaaaggaaagaaaacatctttaaggggaggaaccagagtgctgaaggaatggaagtccatctgcgtgtgtgcagggagactgggtaggaaagaggaagcaaatagaagagagaggttgaaaaacaaaatgggttacttgattggtgattaggtggtggtagagaagcaagtaaaaaggctaaatggaagggcaagtttccatcatctatagaaagctatataagacaagaactcccctttttttcccaaaggcattataaaaagaatgaagcctccttagaaaaaaaattatacctcaatgtccccaacaagattgcttaataaattgtgtttcctccaagctattcaattcttttaactgttgtagaagacaaaatgttcacaatatatttagttgtaaaccaagtgatcaaactacatattgtaaagcccatttttaaaatacattgtatatatgtgtatgcacagtaaaaatggaaactatattgacctaaatgtgaactggttatttctaggtggtgaggtgctttatggtggtgggtttttgctcttgatgccctttttgcattttccaaagtaccatggtgaggatgtgttatatcttttccagggtcctaaaagtccctggcaactccctccccataccctaccatgactggtcacagaaccctttcaccttattgatttgtactgatttcatatggaatatggcaactacatctggctcaaaacaaaggaaaccagaagagccaagtcccaggtgagtgctcagttctgtttctagctttgacgtgtgtgttcttctgtgaaggacaaaatttgcttctattatttaggtaccataatttgtgtttttccaaattaattccctgcag

SEQ ID NO: 2 - protein, 188 aa, Homo sapiens
MDCRKMARFSYSVIWIMAISKVFELGLVAGLGHQEFARPSRGYLAFRDDSIWPQEEPAIRPRSSQRVPPMGIQHSKELNRTCCLNGGTCMLGSFCACPPSFYGRNCEHDVRKENCGSVPHDTWLPKKCSLCKCWHGQLRCFPQAFLPGCDGLVMDEHLVASRTPELPPSARTTTFMLVGICLSIQSYY

SEQ ID NO: 3 - DNA, 24 bp, Artificial
  Synthetic
aagctatggactgcaggaagatgg

SEQ ID NO: 4 - DNA, 22 bp, Artificial
  Synthetic
agaaaggcagatgccaactagc

SEQ ID NO: 5 - DNA, 23 bp, Artificial
  Synthetic
cgccttcggtccagttgccttct

SEQ ID NO: 6 - DNA, 22 bp, Artificial
  Synthetic
atccagattccaagcatccatc

SEQ ID NO: 7 - DNA, 22 bp, Artificial
  Synthetic
atgcgccagaaggcggtatccg

SEQ ID NO: 8 - DNA, 24 bp, Artificial
  Synthetic
ctactcctcatcctcctcactatc

SEQ ID NO: 9 - DNA, 1976 bp, Mus musculus
ggccccatcccctgccggtctacacggagatcttggctgctaacttcccacagactctccaggacgggggcctctctcatttggcatatctttctttttaatctactgttttcactttgtgaaattagcctttgggtgtttcgagaatggctttatgaactaaagccatctgctaatattgtgtttcttgtcttttcctccaacgtttttacgagccgtcgaagatggggtacttctcatccagtgtggttttgcttgtggccatttccagtgcgtttgaatttggacccgttgctgggagagaccttgccatcagagataacagcatttgggaccagaaagaacctgccgtacgcgatcggtctttccagttcgtgccttccgtggggatacagaacagtaagtcgcttaataaaacttgctgtctgaatggagggacttgcatcctggggtccttctgtgcctgccctccttccttctatggacgcaactgtgaacatgatgttcgcaaagagcactgtgggtctatcctccatggcacctggctgcccaagaagtgttccctgtgcagatgctggcacggccagctccactgtcttcctcagacctttctacctggctgtgatggtcacgtgatggaccaggacctcaaagcatccaggactccgtgtcaaacgccgtctgtgacgaccacttttatgctagctggcgcctgcctttttctagatatgaaagtttaggtgtcatgtgaattccatgccagtgccatagcaaagatgtcattcatcttgatgctcacagtgaatccctaatgttacccctcaaaacactaactaggcctttcctctgcacggtccctcctctttctggaaaactatggcgtgtgtgccaagcactgtaacagcgagttacattcctagcctaaaagctactttaagaatgtgctgtctgccatagcctgtgtttcttgatagaagtaactcttacttttcgtcctaagacttttaaacagttctgaaggttattattaaatgccagtgtgcaactggaagtaaattcagagtagctgaaaacagctaaattatctttaagcagggaggtggtggtgtctaccttaaatcaggcaaaggcaggtatatttctgagttccaggacagacaggcctacacagaaaccctgtctcagggaaaaaagagagagagagagagagagagagagagaagaaaaactattatcttaaaagaaaattaatggaccaggcattgtggcagatggctttaatcccagccctcaggaggtagggataggaggatcttcagagttcgaggccagcctggtctatgtagcgaacccctgtctcaaaaaggaaaaagcatcctacgagggagttgaggaagtaatgagggtctgtgcagagggagcagcagatggggaagtgactaagactggggaaacagagtggattgtttgattgatgtttatggaggtactgggaatttaaaaggtaagttcttcagccaagcacagtggtgcacactattaacgtcagctctcaggaggctacaacaggactactaaaagctcatgtccaacctgggctatgtacagagtaccaggccaacaaagactgaatggcaagaccctcacagcaaacccaaacacctgagtctcttttctgcaaaagtacgaaggctttctccaagctatatacaattattcaactgttgtggaggaaagaaagtgtttattattgtagtagtaaaccagattataaaaaccacgtattgtcaagtcagtttttataataattgttcatgaatatgcacagtaaaaatgggaactctgaacaaaaaaaaaaaatgtaaaaaaaggaaagagaaaagaataggaaaagaaggaaggaatccaggtcgcaactgagacgaaaggcggttccagctggcgtgcagatagaaaagtcgtattttaagaacata

SEQ ID NO: 10 - protein, 171 aa, Mus musculus
MGYFSSSVVLLVAISSAFEFGPVAGRDLAIRDNSIWDQKEPAVRDRSFQFVPSVGIQNSKSLNKTCCLNGGTCILGSFCACPPSFYGRNCEHDVRKEHCGSILHGTWLPKKCSLCRCWHGQLHCLPQTFLPGCDGHVMDQDLKASRTPCQTPSVTTTFMLAGACLFLDMKV